(19) United States
(12) Patent Application Publication
(10) Pub. No.: US 2020/0357516 A1
(43) Pub. Date: Nov. 12, 2020

(54) SYSTEMS AND METHODS FOR AUTOMATICALLY INTERPRETING IMAGES OF MICROBIOLOGICAL SAMPLES

(71) Applicant: Beth Israel Deaconess Medical Center, Inc., Boston, MA (US)

(72) Inventors: James E. Kirby, Weston, MA (US); Kenneth P. Smith, Jamaica Plain, MA (US); Anthony Kang, Clarksburg, MD (US)

(21) Appl. No.: 16/762,736

(22) PCT Filed: Nov. 20, 2018
(86) PCT No.: PCT/US18/61946

§ 371 Date: May 8, 2020

Related U.S. Application Data
(60) Provisional application No. 62/589,246, filed on Nov. 21, 2017.

(51) Int. Cl. G16H 50/20; G16H 30/40; G16H 40/60; G06N 3/08; G06K 9/62; G06T 7/00

(52) U.S. Cl. CPC G16H 50/20; G16H 30/40; G16H 40/60; G06T 7/0012; G06K 9/6256; G06K 9/6267; G06N 3/08

(57) ABSTRACT

In accordance with some embodiments of the disclosed subject matter, provided herein are mechanisms (which can, for example, include systems, methods, and media) for automatically interpreting and classifying images of Gram-stained biological samples.

### CROSS-REFERENCE TO RELATED APPLICATIONS

This application claims priority to U.S. Provisional Patent Application No. 62/589,246, filed on Nov. 21, 2017, which is incorporated by reference in its entirety as if fully set forth herein.

### STATEMENT REGARDING FEDERALLY SPONSORED RESEARCH

This invention was made with government support under UL1 TR001102 awarded by the National Institutes of Health and F32 AI124590 awarded by the National Institutes of Health. The government has certain rights in the invention.

### BACKGROUND

Many conditions, such as infections or parasitic infestations, can be diagnosed based on the interpretation of a sample by an expert (e.g., a microbiologist). For example, a sample of blood, tissue, stool, etc., can be obtained from a patient, treated (e.g., by staining the sample using a particular dye or combination of dyes), and viewing the sample microscopically. As a particular example, bloodstream infections (BSI) are rapidly progressive infections with mortality rates up to about 40%, and each day of delay in institution of active antimicrobial therapy for patients that have a BSI is associated with up to about a 10% increase in mortality. Due to relatively low bacterial burden (e.g., <10 colony forming units (CFU) per milliliter (mL), i.e., 10 CFU ml⁻¹), patient blood can be pre-incubated in broth culture to detect presence of bacteria. For example, semi-continuous measurement of carbon dioxide (CO₂) production or pH level with an automated blood culture instrument can be used to detect the presence of organisms. If organism growth is detected (e.g., based on an increase in CO₂ production, a decrease in pH, or oxygen consumption), an aliquot of broth (e.g., now containing >10⁶ CFU mL⁻¹) can removed for Gram stain smear and subculture. In such cases, the Gram stain can provide the first critical piece of information that allows a clinician to tailor appropriate therapy and optimize outcomes in patients with a BSI.

However, despite recent advances in automation in other stages of the BSI diagnosis process (e.g., automated blood culture incubators and Gram staining systems), Gram stain interpretation remains a time consuming and labor intensive process. It can also be highly operator-dependent; proper diagnosis can depend greatly on the skill and judgment of the technician interpreting the results. The Gram stain smear typically provides the first microbiological data to guide treatment for BSI, and earlier results are correlated with positive patient outcome. A recent survey from the American Society for Clinical Pathology indicated that, as of 2014, trained microbiology technologist jobs in the United States had a vacancy rate of ˜9%, and nearly 20% of technologists planned to retire in the next 5 years. This highlights the desirability for development of automated solutions that can allow the current work force to be more efficient.

Although automated slide scanners and microscopes are being used in anatomic pathology (e.g., in telepathology), the application of such scanners in clinical microbiology has been limited due to technical challenges. For example, Gram stained slides are typically read by highly trained microbiologists using 100× objectives, which complicates image acquisition due to the need for addition of oil during scanning. As another example, acceptable images of microbiology smear material can typically be obtained only in a very narrow field of focus, which is not within the capabilities of many existing slide scanners. As yet another example, Gram stained slides exhibit ubiquitous and highly variable background staining, which may cause autofocus algorithms to target areas that are either devoid of bacteria, or miss the appropriate focal plane entirely and instead focus on an area that includes prevalent examples of background staining but few bacteria.

Additionally, image analysis to identify Gram stain characteristics presents other challenges. For example, background and staining artifacts, which are both fairly ubiquitous in Gram stained slides, often mimic the shape and color of bacterial cells. Accordingly, algorithms relying on color intensity thresholding and/or shape detection often provide suboptimal accuracy.

With consolidation of hospital systems, increasing workloads, and potential unavailability of highly trained microbiologists on site, automated image collection paired with computational interpretation of Gram stains to augment and complement manual testing is desirable. Automatic interpretation of other microbiological samples for other conditions presents similar challenges to those described above in connection with interpretation of Gram stained blood samples in the diagnosis of BSIs.

Accordingly, systems, methods, and media for automatically interpreting images of microbiological samples are desirable.

### DETAILED DESCRIPTION

In accordance with various embodiments, mechanisms (which can, for example, include systems, methods, and media) for automatically interpreting images of microbiological samples are provided.

In some embodiments, the mechanisms described herein can use deep learning-based neural networks to automatically interpret Gram stained slides. For example, in some embodiments, an automated imaging process can control an automated slide imaging platform equipped with a 40× air objective to collect highly resolved data from Gram-stained blood culture slides. In some embodiments, image data representing pre-classified Gram-stained blood culture slides can be used to train a deep-learning neural network, such as a convolutional neural network (CNN)-based model, to recognize morphologies representing common causative agents of BSI, such as Gram-negative rods, Gram-positive cocci in clusters, and Gram-positive cocci in pairs or chains. In general, CNNs are modeled based on the organization of neurons within the mammalian visual cortex. In some embodiments, the mechanisms described herein can use a CNN for its ability to excel in image recognition tasks, without requiring time-intensive selective feature extraction by humans.

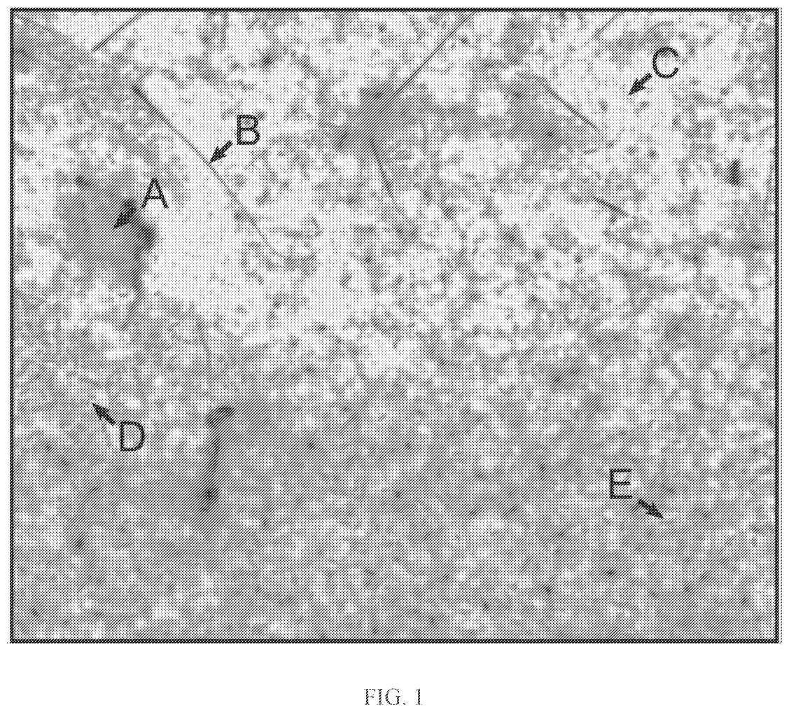

FIG. 1 shows an example of image data representing a portion of a Gram-stained blood culture slide. The image in FIG. 1 includes many features typical of blood culture Gram stain smears including: (A) intense background staining; (B) a stain crystallization artifact; (C) diffuse background staining; (D) individually resolvable, high-contrast Gram-negative cells; and (E) individually resolvable, low-contrast Gram-negative cells. Note that ubiquitous background material is often similar in color, intensity, and/or shape to bacterial cells, as shown in FIG. 1.

While highly experienced medical technologists can readily differentiate bacteria from this background, manually defining computational rules for Gram-stain classification that would adequately distinguish signal from noise in highly variable Gram-stain preparations would be an extremely complex, and potentially impossible, task. By contrast, CNNs do not require manual definition of rules. CNNs generally consist of a number of layers of computation cells, with certain cells/layers convoluting regions of the image to detect specific features, and other cells/layers condensing the information (e.g., using a Max Pooling, Average Pooling, or other rule). The parameters used in various layers to transform the underlying data can be trained such that the CNN can effectively recognize particular classes of objects which may be very diverse (e.g., birds, chairs, cars, dishwashers, zebras, etc.), or may be superficially similar to one another although they belong to distinct classes (e.g., different types of flowers). During each phase of the training process, a subset of pre-classified images can be presented to the network, which can be used to set the values of various parameters, such that the CNN automatically identifies features important for classification based on, for example, optimization of output accuracy. A trained CNN can be defined by a set of weights and biases that control the flow of information through the network such that the most discriminatory features in the images are used for classification.

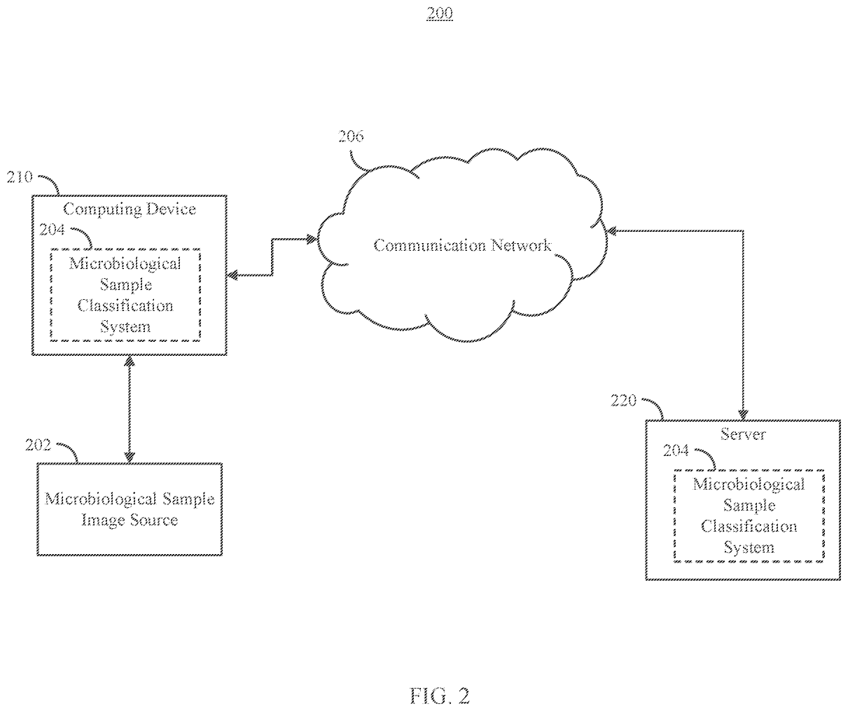

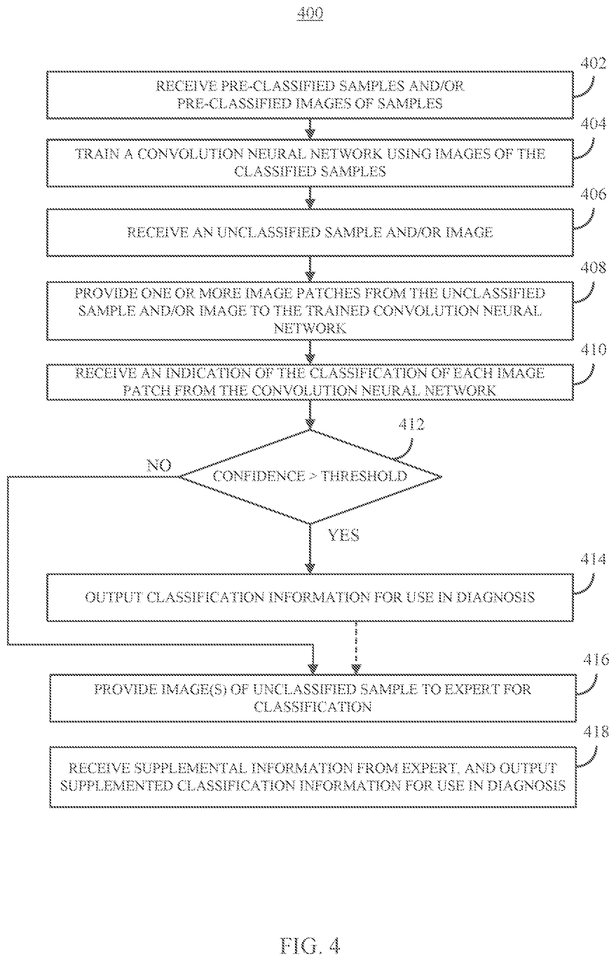

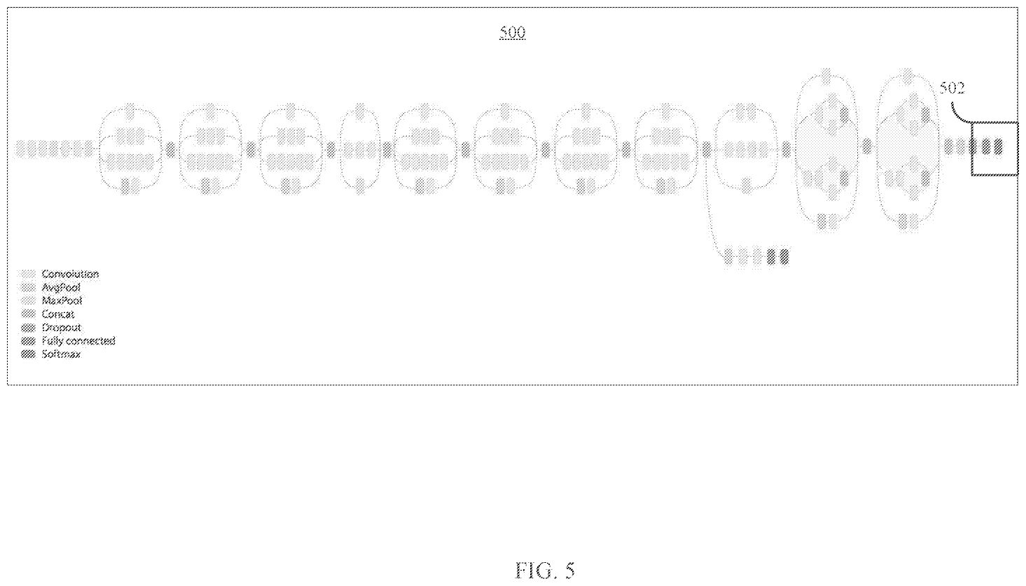

Turning to FIG. 2, an example 200 of a system for automatically interpreting images of microbiological samples is shown in accordance with some embodiments of the disclosed subject matter. As shown in FIG. 2, a computing device 210 can receive one or more images of a microbiological sample from a microbiological sample image source 202. In some embodiments, computing device 210 can execute at least a portion of a microbiological sample classification system 204 to classify the microbiological sample based on one or more images of the sample received from image source 202. Additionally or alternatively, in some embodiments, computing device 210 can communicate information about the one or more images of the sample received from image source 202 to a server 220 over a communication network 206, and server 220 can execute at least a portion of microbiological sample classification system 204 to classify the microbiological sample based on one or more images of the sample. In some such embodiments, server 220 can return information to computing device 210 (and/or any other suitable computing device) indicative of an output of microbiological sample classification system 204, such as an indication that the presence of Gram-negative cells has been detected, a confidence associated with the determination that Gram-negative cells are present, one or more images that were ranked by the classification system as the most likely to contain Gram-negative cells, etc. In some embodiments, computing device 210 and/or server 220 can be any suitable computing device or combination of devices, such as a desktop computer, a laptop computer, a smartphone, a tablet computer, a wearable computer, a server computer, a virtual machine being executed by a physical computing device, a special purpose computer (e.g., a computer integrated into a medical device), etc. In some embodiments, computing device 210 and server 220 can execute different CNNs. For example, computing device 210 can execute a first CNN, and server 220 can execute a second CNN that uses more parameters, more layers, and/or is otherwise more computationally intensive to execute. In such an example, if the first CNN executed by computing device 210 does not make a prediction with sufficient confidence (e.g., as described below in connection with FIG. 4), the second CNN executed by server 220 can be used to classify the sample. Additionally or alternatively, in some embodiments, server 220 (which can be any suitable number of servers, e.g., configured as part of a cloud computing system) can execute both the first CNN and second CNN, and can interpret more samples with more accuracy than if just the first CNN or second CNN alone were used, as the first CNN may be able to classify samples relatively quickly (e.g., because it is less computationally intensive), and the second CNN can classify any samples that are more ambiguous or otherwise more difficult for the first CNN to classify. In some embodiments, multiple CNNs can be used to recognize different types of organisms. For example, a first CNN can determine whether relatively rare bacteria are likely to be present in a blood culture Gram stain slide, and depending on the classification, the sample can be analyzed by either a second CNN that is trained to recognize relatively common types of bacteria, or a third CNN that is trained to recognize relatively rare types of bacteria. As described below in connection with FIGS. 4 and 5, microbiological sample classification system 204 can use one or more trained convolution neural networks to determine whether a particular pathogen or one or more indicators of a particular condition is present in a microbiological sample, and can present information about the determination to a user (e.g., a physician, a physician's assistant, a nurse practitioner, a nurse, a technician, a microbiology technologist, a pharmacist, etc.).

In some embodiments, image source 202 can be any suitable source of images of a microbiological sample, such as a manually or semi-manually operated microscope (e.g., a manually operated microscope configured with a 100× oil objective) in communication with computing device 210, a completely automated slide scanning system (e.g., including a microscope configured with a 40× dry objective) in communication with computing device 210, another computing device (e.g., a server storing one or more images), etc. In some embodiments, image source 202 can be local to computing device 210. For example, image source 202 can be incorporated with computing device 210 (e.g., computing device 210 can be configured as part of a device for capturing, scanning, and/or storing images of microbiological samples). As another example, image source 202 can be connected to computing device 210 by a cable, a direct wireless link, etc. Additionally or alternatively, in some embodiments, image source 202 can be located locally and/or remotely from computing device 210, and can communicate images of microbiological samples to computing device 210 (and/or server 220) via a communication network (e.g., communication network 206).

In some embodiments, communication network 206 can be any suitable communication network or combination of communication networks. For example, communication network 206 can include a Wi-Fi network (which can include one or more wireless routers, one or more switches, etc.), a peer-to-peer network (e.g., a Bluetooth network), a cellular network (e.g., a 3G network, a 4G network, etc., complying with any suitable standard, such as CDMA, GSM, LTE, LTE Advanced, WiMAX, etc.), a wired network, etc. In some embodiments, communication network 206 can be a local area network, a wide area network, a public network (e.g., the Internet), a private or semi-private network (e.g., a corporate, hospital, diagnostic laboratory, or university intranet), any other suitable type of network, or any suitable combination of networks. Communications links shown in FIG. 2 can each be any suitable communications link or combination of communications links, such as wired links, fiber optic links, Wi-Fi links, Bluetooth links, cellular links, etc.

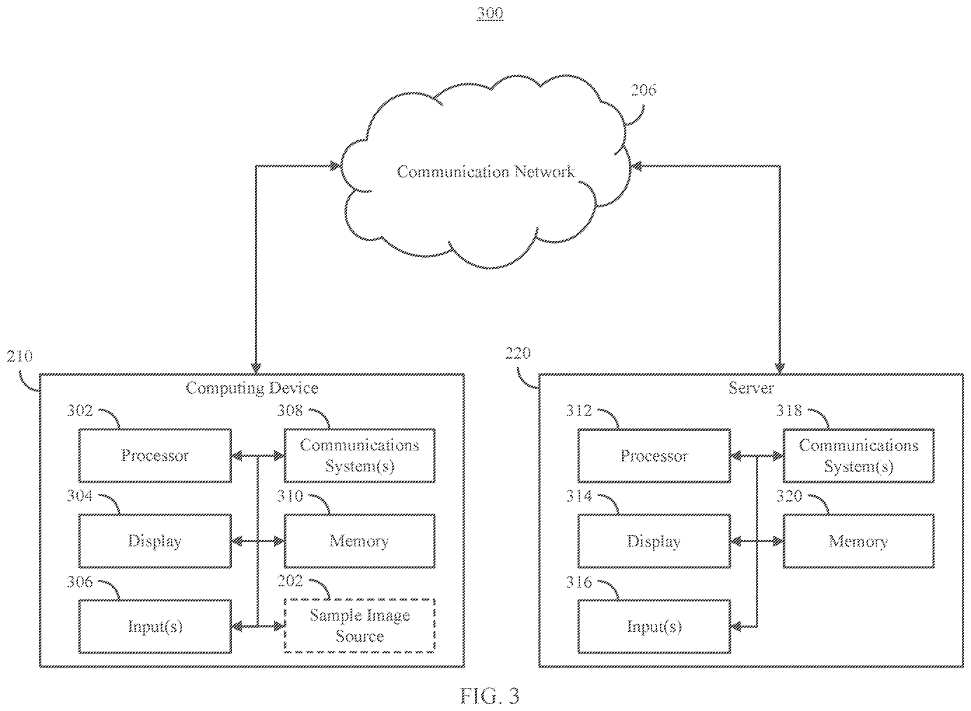

FIG. 3 shows an example 300 of hardware that can be used to implement computing device 210 and server 220 in accordance with some embodiments of the disclosed subject matter. As shown in FIG. 3, in some embodiments, computing device 210 can include a processor 302, a display 304, one or more inputs 306, one or more communication systems 308, and/or memory 310. In some embodiments, processor 302 can be any suitable hardware processor or combination of processors, such as a central processing unit (CPU), a graphics processing unit (GPU), etc. In some embodiments, display 304 can include any suitable display device(s), such as a computer monitor, a touchscreen, a television, etc. In some embodiments, inputs 306 can include any suitable input device(s) and/or sensor(s) that can be used to receive user input, such as a keyboard, a mouse, a touchscreen, a microphone, etc.

In some embodiments, communications system(s) 308 can include any suitable hardware, firmware, and/or software for communicating information over communication network 206 and/or any other suitable communication networks. For example, communications system(s) 308 can include one or more transceivers, one or more communication chips and/or chip sets, etc. In a more particular example, communications system(s) 308 can include hardware, firmware and/or software that can be used to establish a Wi-Fi connection, a Bluetooth connection, a cellular connection, an Ethernet connection, etc.

In some embodiments, memory 310 can include any suitable storage device or devices that can be used to store instructions, values, etc., that can be used, for example, by processor 302 to present content using display 304, to communicate with server 220 via communications system(s) 308, etc. Memory 310 can include any suitable volatile memory, non-volatile memory, storage, or any suitable combination thereof. For example, memory 310 can include RAM, ROM, EEPROM, one or more flash drives, one or more hard disks, one or more solid state drives, one or more optical drives, etc. In some embodiments, memory 310 can have encoded thereon a computer program for controlling operation of computing device 210. In some such embodiments, processor 302 can execute at least a portion of the computer program to present content (e.g., images, user interfaces, graphics, tables, etc.), receive content from server 220, transmit information to server 220, execute at least a portion of a microbiological sample classification program, etc.

As described above, in some embodiments, image source 202 can be incorporated into computing device 210. Additionally or alternatively, computing device 210 can communicate with image source 202 using communications system(s) 308 and/or any using any other suitable communication hardware, such as via a USB cable, etc.

In some embodiments, server 220 can include a processor 312, a display 314, one or more inputs 316, one or more communications systems 318, and/or memory 320. In some embodiments, processor 312 can be any suitable hardware processor or combination of processors, such as a central processing unit, a graphics processing unit, etc. In some embodiments, display 314 can include any suitable display device(s), such as a computer monitor, a touchscreen, a television, etc. In some embodiments, inputs 316 can include any suitable input devices and/or sensors that can be used to receive user input, such as a keyboard, a mouse, a touchscreen, a microphone, etc.

In some embodiments, communications system(s) 318 can include any suitable hardware, firmware, and/or software for communicating information over communication network 206 and/or any other suitable communication networks. For example, communications system(s) 318 can include one or more transceivers, one or more communication chips and/or chip sets, etc. In a more particular example, communications system(s) 318 can include hardware, firmware and/or software that can be used to establish a Wi-Fi connection, a Bluetooth connection, a cellular connection, an Ethernet connection, etc.

In some embodiments, memory 320 can include any suitable storage device or devices that can be used to store instructions, values, etc., that can be used, for example, by processor 312 to present content using display 314, to communicate with one or more computing devices 210, etc. Memory 320 can include any suitable volatile memory, non-volatile memory, storage, or any suitable combination thereof. For example, memory 320 can include RAM, ROM, EEPROM, one or more flash drives, one or more hard disks, one or more solid state drives, one or more optical drives, etc. In some embodiments, memory 320 can have encoded thereon a server program for controlling operation of server 220. In such embodiments, processor 312 can execute at least a portion of the server program to transmit information and/or content (e.g., results of a bone age assessment, a user interface, etc.) to one or more computing devices 210, receive information and/or content from one or more computing devices 210, receive instructions from one or more devices (e.g., a personal computer, a laptop computer, a tablet computer, a smartphone, etc.), execute at least a portion of a microbiological sample classification program, etc.

FIG. 4 shows an example 400 of a process for training a CNN and classifying a microbiological sample in accordance with some embodiments of the disclosed subject matter. As shown in FIG. 4, at 402, process 400 can receive examples of pre-classified samples and/or pre-classified images of samples. In some embodiments, the pre-classified samples and/or images of samples can be received in any suitable condition and/or from any suitable source. For example, in some embodiments, the pre-classified samples can be samples generated during the course of normal operations of a laboratory. In such an example, during its normal course of operations a laboratory can receive a sample (e.g., a blood sample, a tissue sample, a stool sample, etc.) to be analyzed for the presence of a particular condition(s) and/or pathogen(s). The sample as received may already be prepared for analysis (e.g., a facility that collected the sample may have also prepared the sample for analysis, by, for example, applying a stain, placing the sample on a slide, etc.), or may be prepared for analysis by the laboratory. In such an example, the sample can be examined, and a determination can be made about whether the particular condition(s) and/or pathogen(s) is/are present or absent. As part of its operations, the laboratory may store the prepared sample and/or images captured of the sample during analysis for its records, with an indication of the result of the analysis (e.g., whether a particular condition(s) and/or pathogen was found to be present, a level or count of the pathogen, etc.). In some embodiments, process 400 can receive such archived samples and/or images of samples, and the indication of whether a particular condition and/or pathogen was present.

In a more particular example, blood culture Gram stain slides that were prepared manually during the course of normal laboratory operation can be used for analysis. Such slides can be selected based on the presence of any of the three most common morphotypes observed in bloodstream infection: Gram-positive cocci in clusters, Gram-positive cocci in pairs and chains, and Gram-negative rods. Additionally or alternatively, in some embodiments, slides can be selected based on less common morphotypes (e.g., Gram-positive rods or yeast) and/or polymicrobial infections. As samples prepared by technologists during the course of customary laboratory operation are likely to vary significantly, in some embodiments, slides can be selected without pre-screening for suitability for automated microscopy or deep learning. Accordingly, in such an example, the samples and/or images of blood culture Gram stain slide received at 402 can have slide-to-slide variability in staining intensity, staining artifacts, and sample distribution. Such variability in training data may result in more robust slide classification models due to the potentially wide variability in conditions.

Additionally or alternatively, in some embodiments, samples can be collected, imaged, and classified for the purpose of generating data that can be used to train a classification model.

In general, CNN-based deep learning models exhibit better performance with large datasets for training, typically at least on the order of thousands of images (and may continue to benefit with increasing numbers of samples, for example, using tens of thousands of images may result in better performance than using thousands of images).

In some embodiments, automated microscopy image acquisition techniques can be used to capture images of samples for use in training the CNN, as this can generate a relatively large number of images relatively efficiently as compared to manual image acquisition. For example, a MetaFer Slide Scanning and Imaging Platform (available from MetaSystems Group, Inc., of Newton, Mass.), which can be configured with a Gram stain-compatible autofocus system, can sample multiple distributed positions on a slide, and can be configured with an automated slide loading system. In such an example, the MetaFer Slide Scanning and Imaging Platform can capture images at distributed positions, which can capture images with meaningful data from slides with variations in specimen distribution, and can enable relatively high throughput slide scanning.

In general, in a clinical setting Gram stains are read under oil immersion using a microscope with a 100× oil objective. However, semi-continuous addition of oil during automated microscopy may be undesirable and/or inefficient. Accordingly, in this more particular example, images of samples from uncoverslipped slides can be captured with a 40× dry objective, which can provide sufficient resolution for machine-learning. Using the lower powered dry objective can avoid the need for oil immersion, and facilitate capture of a larger field of view in each image. In such an example, the MetaFer Platform can be used to capture a relatively large number of unique images that can be used for training a CNN from a relatively low number of slides. In one particular example, a total of 25,488 images were automatically collected from distributed locations on 180 slides.

In some embodiments, any suitable number of images can be captured from a slide/sample. For example, in some embodiments, the entire area of the slide at one or more depths of field can be captured using an automated or semi-automated process. As another example, images can be captured at various positions of a slide/sample. In such an example, the images can be captured at the same pre-defined positions on every slide, with enough images captured that it is expected that any pathogens on the slide would be present in at least a portion of the images. Additionally or alternatively, the imaging platform, a computing device controlling its operation, and/or a user supervising its operation, can select positions on a slide that are most likely to include relevant information. In a more particular example, an automated rapid scan can be performed by the imaging platform, a computing device controlling its operation, and/or a user supervising its operation for areas of appropriate staining intensity, which can be selected for imaging. In some embodiments, such affirmatively selected positions can be captured in addition to images captured at pre-selected positions, or only images captured at the affirmatively selected positions can be used.

In some embodiments, further processing can be performed on the captured images to produce yet more examples that can be used for training the CNN. For example, relatively small patches of image data can be extracted from automatically captured images. Such image patches can be any suitable size. In one particular example, the image patches can be 146×146 pixels. In some embodiments, the relatively small patches can be selected automatically. For example, by analyzing the image for areas that exhibit certain characteristics. In some embodiments, the relatively small patches can be selected semi-automatically. For example, small patches from the images can be extracted automatically and presented to a user, and the user can provide input indicating whether the patch includes at least a threshold amount of subject matter that is not background (e.g., that the patch likely includes a positive example of the class), or that the patch is background. As another example, at least a portion of an image can be presented to a user (e.g., by computing device 210), and the user can select points in the image that include an example of a particular class (e.g., using a mouse, using a touchscreen, etc.). In a more particular example, a selection tool implemented using the Python programming language can be executed by a computing device that presents an image containing bacteria, and a user can select portions of the image that include bacteria with a single mouse click, and a patch around the location of the mouse click can be copied and saved as a positive example of whatever class the image belongs to (e.g., if the slide was an example of a sample with Gram-negative rods, the patch can be labeled as an example including Gram-negative rods). The user can also select areas of each image that do not include bacteria, but include staining and/or other artifacts, and the selection tool can label patches as positive examples of background.

In general, blood culture Gram stain slides include mostly background (i.e., a majority of the area of the slide does not include concentrations of bacteria). Using many images that include such background as positive examples of a particular class may increase the chance that a CNN trained using that data will learn features that are unrelated to bacterial Gram-stain classification. This may result in overfitting, such that the model trained using such data has high accuracy in classifying images used for training (i.e., the training set), but relatively worse accuracy when presented with a validation set, or an unknown sample. Accordingly, in some embodiments, the training data can be enriched through use of more carefully selecting only portions of the images rather than whole slide images or relatively large images from a portion of the slide. In some embodiments, any suitable number of image patches can be generated for training the CNN. In one particular example, a selection tool was used to generate a total of 100,213 manually classified 146×146 pixel image patches from 25,488 images captured of samples included on 180 slides.

At 404, process 400 can train a CNN using the images received and/or generated at 402. In some embodiments, any suitable CNN can be trained using any suitable technique or combination of techniques. For example, in some embodiments, a relatively complex CNN such as the Inception v3 (an example of which is shown in FIG. 5) can be trained to interpret images of microbiological samples. In such an example, the entire Inception v3 CNN can be trained using the images received at 402. In a more particular example, the Inception v3 CNN can be trained to perform complex image classification tasks, such as relatively accurate classification of 1,000 different objects, as described, for example, in Szegedy et al., “Rethinking the Inception Architecture for Computer Vision,” available at https(colon)//arxiv(dot)org/abs/1512.00567 as of Sep. 12, 2017, which is hereby incorporated by reference herein in its entirety. In general, the Inception v3 CNN includes a series of smaller convolutional networks which are sometimes referred to as “inception modules,” and it was designed to be less computationally intensive than other peer CNNs. As described above, the entire Inception v3 CNN can be trained, however, this may require weeks of computing time even with state-of-the-art computational infrastructure.

Alternatively, in some embodiments, one or more transfer learning techniques can be used to retrain only a portion of an already trained example of the Inception v3 CNN. For example, many image classification tasks can be addressed using pre-computed parameters from a version of the Inception v3 CNN that was trained to classify an unrelated image set. In one particular example, a version of the Inception v3 CNN trained to recognize 1,000 different image classes from the 2012 ImageNet Large Scale Visual Recognition Competition dataset can be retrained using transfer learning techniques to generate new parameters for the final fully connected layer to identify Gram stain categories of interest, rather than the 1,000 different image classes. In such an example, the output layer (e.g., a Softmax layer in the Inception v3 CNN) can be configured to output the probability assigned by the CNN for each of various classes. As shown in FIG. 5, the final fully connected layer and softmax layer shown in Box 502 can be modified in a transfer learning process, and the remainder of the Inception v3 CNN can retain the parameters generated during training on the independent image set, which can be used to perform feature extraction.

Note that although the Inception v3 CNN is described herein, this is merely an example and many other types of CNNs can be used, such as AlexNet, GoogleNet, VGGNet, etc. Note that each of various CNNs have a unique architecture that may differ in organization, function and/or the number of convolutional layers compared to the Inception v3 CNN, and other CNNs.

In some embodiments, the CNN trained at 404 can be configured to output relative probabilities that an image input to the CNN belongs to each of various classes. For example, the CNN can be configured to output the relative probability that the image includes Gram-positive cocci in chains/pairs, Gram-positive cocci in clusters, Gram-negative rods, and background (i.e., no bacteria). In some embodiments, the class with the highest relative probability can be assigned as the predicted class.

In some embodiments, the images received and/or generated at 402 can be subdivided into a training set, a validation set, and a test set. The CNN can be trained using the training set, and its accuracy after one or more training iterations, the accuracy of the CNN can be checked using images from the validation set. When the accuracy (or another suitable metric) stops increasing/changing by more than a threshold amount between iterations, training can be stopped and the CNN can be evaluated using the test set that was withheld and not used in either the training set or validation set.

At 406, process 400 can receive an unclassified sample and/or image to be interpreted using the CNN trained at 404. Note that, a computing device that is configured to use to the CNN trained at 404 may or may not be the same computing device that was used to train the CNN at 404. For example, the CNN can be trained by a first computing device and characteristics of the trained CNN, such as the values of parameters and/or configuration information of the CNN (e.g., the number of layers, the interconnection of the layers, etc.) can be saved. At least a portion of the characteristics can then be communicated to any suitable number of other computing devices, which can use the characteristics to execute a copy of the trained CNN. That is, training at 404 can be executed by a first computing device, and receiving the unclassified sample and/or image to be interpreted at 406 can be executed by another computing device at any suitable later time and/or any suitable location (e.g., the second computing device can be remote from the computing device that trained the CNN at 404).

In some embodiments, one or more image patches can be extracted from one or more images captured of the sample. For example, if a slide including the sample if available, one or more images can be captured from the slide, and each of those images can be subdivided into relatively small patches (e.g., the same size of image patch that was used in training the CNN, such as a 146×146 pixel patch), which can each be input into the trained CNN. In a more particular example, images can be captured at pre-defined positions of the slide and/or at positions of the slide that are determined (e.g., by an imaging platform) to be most likely to include positive examples (i.e., not background) of one or more of the classes that the CNN is trained to detect.

As another example, if one or more images of a sample are received (e.g., from a remote laboratory), each of those images can be subdivided into relatively small patches (e.g., 146×146 pixel patches), which can each be input into the trained CNN.

In some embodiments, any suitable number of images and/or image patches can be classified per sample. For example, in a fully automated system, a relatively large number of image patches can be extracted to try to ensure that any positive examples of a non-background class from the sample are included in at least one patch. As another example, in a semi-automated system, a user can identify positions in the one or more images of the sample that are most likely to include a positive example of at least one non-background class. For example, a selection tool can be executed by a computing device executing at least a portion of process 400 that can allow a user to view images of the sample and select one or more positions that appear most likely to include a positive example of at least one non-background class.

At 408, each of one or more image patches depicting a portion of the unclassified sample can be provided as input to the trained CNN. Note that, in some embodiments, the trained CNN can be provided with the image patches in a serial fashion, and the CNN can analyze each image patch in turn. Additionally or alternatively, the image patches can be grouped into batches, and can be evaluated in parallel by copies of the trained CNN.

At 410, process 400 can receive an indication of the classification of each image patch from the trained CNN. For example, as each image patch is evaluated, the trained CNN can provide the probability/confidence that the patch includes an example of each of various classes. In a more particular example, the trained CNN can provide the probability/confidence that the patch includes Gram-positive cocci in chains/pairs, Gram-positive cocci in clusters, Gram-negative rods, and background.

In some embodiments, process 400 can count the number of patches that are predicted to belong to each class, and can classify the sample based on the class that has the highest count. The trained CNN can provide all probabilities, can provide all probabilities over a particular threshold, or can provide only the predicted class and a probability associated with the predicted class.

In some embodiments, process 400 can count a patch as a particular class if the confidence associated with the prediction is greater than a threshold confidence. For example, if the confidence is at least 50%, 75%, 90%, 99%, 99.9%, or any other suitable value, that the image patch that was evaluated by the trained CNN belongs to the predicted class, process 400 can count the image patch as an example of the predicted class. Otherwise, if the confidence is less than or equal to the threshold, process 400 can count the patch as belonging to a different class, which may or may not be a class that the trained CNN was trained to recognize. For example, when evaluating blood culture Gram stained slides, process 400 can count any image patches that did not meet the threshold as being background images, as such a sample is expected to include bacteria (as the sample is only analyzed if the CFU is expected to be at least a threshold value, such as at least 10⁶ CFU ml⁻¹). However, for different pathogens where the result may be positive or negative, process 400 can count image patches that did not meet the threshold as being an unknown class, unclassified, or some other category that indicates that there was not enough information to make a positive determination of whether there is a positive example of a particular class in the patch.

In some embodiments, process 400 can label the sample based on the results of the individual analyses of the one or more image patches. For example, in some embodiments, process 400 can label the sample based on which class has the highest count. As another example, process 400 can label the sample based on which class has the highest count only if that count is at least a threshold number. As yet another example, process 400 can label the sample based on any class (other than background/negative) that has a count that is at least a threshold number (e.g., at least 100 image patches belonged to a particular class).

In some embodiments, process 400 can determine the confidence that the sample includes a particular class and/or should be labeled as belonging to a particular class based on any suitable information. For example, in some embodiments, process 400 can determine the confidence based on the count for the class with the highest count compared to the total number of image patches that were analyzed. As another example, in some embodiments, process 400 can determine the confidence based on the count for the class with the highest count compared to the total number of image patches that were not labeled as background. As yet another example, in some embodiments, process 400 can determine the confidence based on the count for the class with the highest count compared to the total number of image patches that were labeled as background. As still another example, in some embodiments, process 400 can determine the confidence based on the ratio of the count for the class with the highest count to another class or classes (e.g., the number of image patches that fell below the confidence threshold for an individual image patch classification).

At 412, process 400 can determine whether the confidence in the classification of the sample is greater than a threshold. If process 400 determines that the confidence is greater than the threshold (“YES” at 412), process 400 can move to 414, and can output classification information for the sample for use in diagnosis or treatment of the patient from which the sample was taken. Additionally, in some embodiments, one or more images and/or image patches that were classified as including a positive example of the class matching the ultimate classification of the sample can be included. Additionally or alternatively, in some embodiments, process 400 can move to 416 (as described below) for expert confirmation and/or verification of the classification. In some such embodiments, process 400 can move to 416 from 414 when the confidence is above the threshold, but below a second threshold, when the classification is of the sample is relatively uncommon (e.g., the classification is background when a positive result is expected, the organism that is predicted to be present is unexpected in the patient from which the sample was taken, etc.).

Otherwise, if process 400 determines that the confidence is not greater than the threshold (“NO” at 412), process 400 can move to 416. At 416, process 400 can provide one or more images and/or image patches of the unclassified sample to an expert (e.g., a highly trained microbiologist) for classification. In some embodiments, images that included patches that were classified as a particular class (e.g., the class with the highest count value) can be presented to the expert, and the expert can provide input indicating whether they agree with the classification of the sample, disagree with the classification, are unsure whether the classification is correct, etc. In some embodiments, process 400 can provide the classification by the trained CNN to the expert with or without the confidence in the classification. Alternatively (e.g., to avoid biasing the expert), process 400 can present the images without the classification by the CNN, and the classification by the expert can be compared to the classification by the trained CNN. In some embodiments, images and/or patches that fell below the confidence threshold for individual image patches (e.g., the trained CNN was not able to make a positive classification that a particular class was represented with enough confidence) can be presented to the expert, and the expert can classify one or more of the images and/or patches as positively including a particular pathogen, or as not including any pathogens of interest (e.g., background).

At 418, process 400 can receive supplemental information generated based on the expert's input, and can output supplemented classification information for use in diagnosis and/or treatment of the patient from which the sample was taken. In some embodiments, such supplemented classification information can include only the expert's classification, both the expert's classification and the classification by the trained CNN, a classification without an indication of whether the classification was made by an expert or by a trained CNN, etc. Additionally, in some embodiments, one or more images and/or image patches that were classified as including a positive example of the class matching the ultimate classification of the sample can be included.

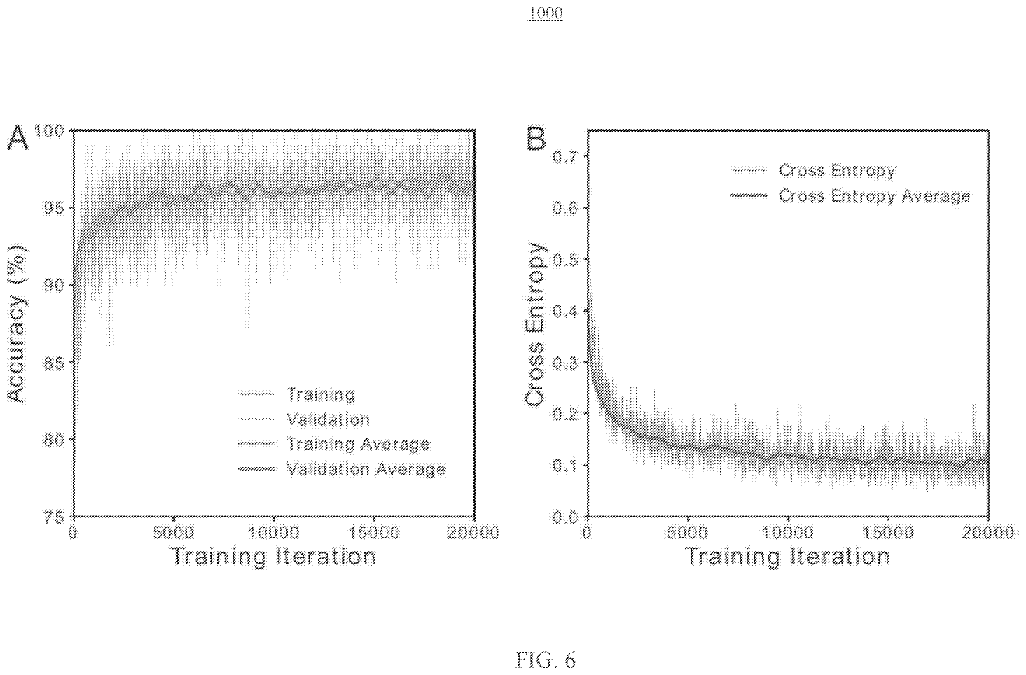

FIG. 6 shows an example of accuracy and cross entropy values over a range of training iterations for a version of the Inception v3 CNN that was trained using transfer learning techniques to recognize the presence of various bacteria in blood culture Gram stain slides in accordance with some embodiments of the disclosed subject matter. The results in FIG. 6 were generated from an Inception v3 CNN trained using transfer learning from a training set of 100,213 manually classified image patches of 146×146 pixels each. These image patches were selected from images collected from distributed locations on 180 blood culture Gram stain slides that were prepared manually during the course of normal laboratory operation. In this manner, training and implementation of the CNN can be implemented using image patches (sometimes also referred to as image “crops”). A tool for selecting and extracting 146×146 image patches from the images was used to generate images to be used to train, validate, test, and evaluate the CNN. The selection tool was implemented using the Python programming language, and allowed a user to select areas of an image containing bacteria with a single input (e.g., mouse click), and extracted an image patch from around the position that was selected. The CNN was trained to predict whether an image is an example of one of four classes: Gram-positive cocci in chains/pairs; Gram-positive cocci in clusters; Gram-negative rods; and background (i.e., not belonging to any of the other three classes).

As shown in FIG. 6, training and validation accuracy were highly correlated, which may be an indication that the trained CNN is generalizable for novel images that were not used in the training process. During training, predictions made by the CNN were compared to the observed data, and differences between the values were quantified using a metric that is sometimes referred to as cross-entropy, which is further described in de Boer, et al., “A Tutorial on the Cross-Entropy Method,” Annals of Operations Research 134:19-67 (2005). In general, low cross-entropy may indicate that a particular model fits the observed data well. As shown in FIG. 6, cross entropy decreased during training and plateaued after about 12,000 iterations. Additional training iterations beyond those shown in FIG. 6 did not significantly reduce cross-entropy or improve accuracy.

After training, a classification accuracy of 94.9% was observed for the trained CNN when tested using a set of test images that were not used during the training phase. However, the accuracy on the test set may not reflect the true accuracy of the trained CNN for entirely novel samples, as the test set was not wholly independent of the training set (e.g., it may have included image patches from the same slide and/or images as image patches in the training and validation sets). That is, certain images in the training set may have come from the same sample, although they did not depict the same portion of the sample.

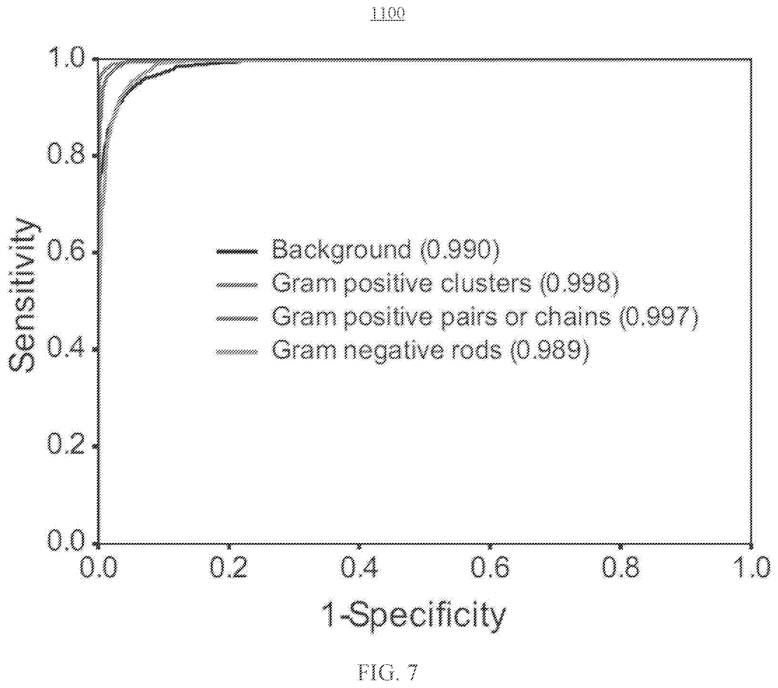

Classification accuracy of the CNN was also evaluated based on an evaluation set of 4,000 manually classified image patches (with 1,000 image patches representing each of the four class) from 59 slides that were not used to generate images that were included in the training set, validation set, or test set. A classification accuracy of 93.1% was observed for the evaluation set. Additionally, classifications of the evaluation set facilitated calculation of sensitivity and specificity on a per-category basis. Observed sensitivity/specificity was 96.6/99.4% for Gram-positive clusters, 97.7/99.0% for Gram-positive chains, 80.1/99.4% for Gram-negative rods, and 97.4/93.0% for background. FIG. 7 shows examples of receiver operating characteristic (ROC) curves, and the area under the ROC curve (AUC) for each category may indicate the ability of the trained CNN to differentiate between categories (AUC>0.98 for all categories).

The observed accuracies of the trained CNN described above in connection with FIG. 6 were based on manually selected image patches based on category assignment using the highest probability output from the classification. However, this may not be an appropriate technique when attempting to perform a classification for a sample (e.g., a whole slide), rather than a single image patch. More particularly, a whole sample classification task can differ from the above-described example, as making an accurate classification of a sample may involve generating classifications for a relatively large number of individual image patches that are automatically selected, many of which may be background (i.e., contain no bacteria). As described above in connection with FIG. 1, background may simulate bacterial cells. Accordingly, when many automatically selected image patches of background are evaluated, there may be a relatively high false positive rate when the proper classification for those image patches is background.

In one example, a relatively large threshold of 0.99 was set for confidence required to predict membership of an image patch in a class, which may reduce false positives at the image patch level, and maximize specificity at the whole sample level. That is, if the trained CNN indicated that a particular image patch was a member of a particular class (other than background), but with a probability of less than 99%, that patch was classified as being background. Note that this may not be an appropriate classification when evaluating a sample for the presence of a different pathogen and/or condition, as the blood culture Gram stain slides are expected to include bacteria. For other pathogens, such as certain fungi or parasites, where the classification may be for the purpose of determining whether the pathogen is present (and in some cases a class of fungus or parasite that is present), not merely determining which class is present assuming that at least one class of bacteria is present. Using this stringent cutoff, 65.6% of evaluated image patches had a prediction with confidence of ≥0.99, and 99.6% of those were correctly classified (i.e., of the 65.6% of images, only 0.4% were misclassified). Observed classification accuracy was 99.9% for Gram-positive clusters, 100% for Gram-positive chains, and 97.4% for Gram-negative rods. The other 34.4% of evaluated image patches had a prediction with confidence of <0.99.

Additionally, in another example, 350 whole images containing no visible bacteria cells which were not part of the training set, validation set, test set, or evaluation set. The images were subdivided into 192 non-overlapping 146×146 pixel patches (for a total of 67,200 image patches) using a Python script. Each image patch was evaluated by the trained CNN with a 0.99 classification threshold. For each category, the false positive rate was ≤0.006% on a per image patch basis (i.e., for each class corresponding to the presence of a class of bacteria, less than 0.006% of the image patches that were known to contain no bacteria were misclassified with at least a 99% confidence as belonging to that non-background class). Based on an assumed normal distribution of false positives classifications, a minimal threshold for sample classification of 6 positive image patches per category to achieve an expected false positive sample classification rate of ≤0.1%.

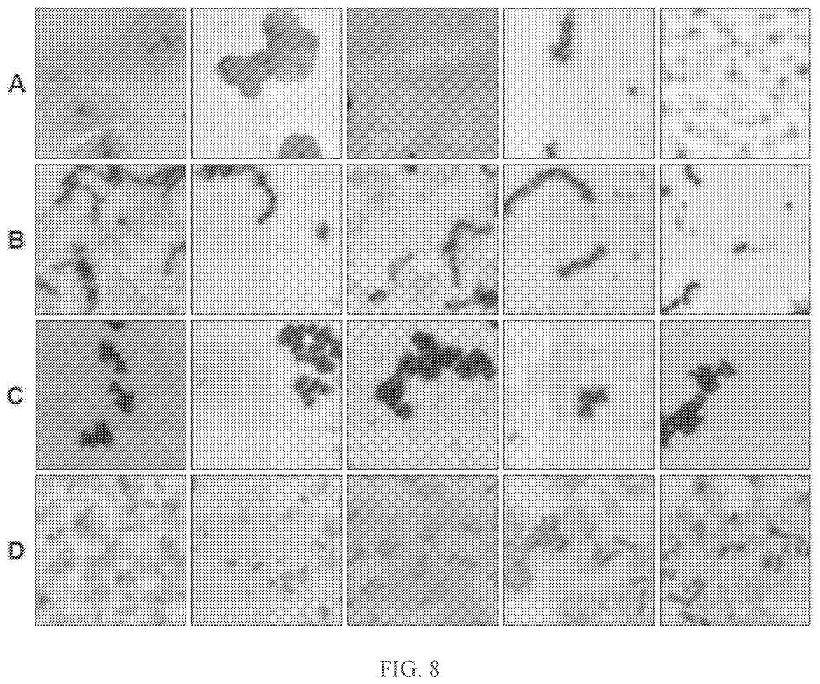

In an example, a whole sample classification technique was used to classify 189 samples (with one sample being represented by one slide). The technique included capturing 54 images of each slide at pre-defined positions, dividing each image into 192 non-overlapping 146×146 pixel image patches, evaluating each individual image patch using the trained CNN, and counting the number of image patches classified as each class corresponding to a positive detection of a particular type of bacteria (i.e., Gram-positive cocci in chains/pairs, Gram-positive cocci in clusters, and Gram-negative rods), for a total of 10,368 image patches (and data points) per slide. A selection of image patches corresponding to the various different categories were presented for manual review. FIG. 8 shows examples of image patches there were correctly classified during a whole sample evaluation test using the trained CNN. In FIG. 8, each image represents a correctly classified image patch that was automatically extracted from an image during whole sample (whole slide) classification, with row (A) includes image patches correctly classified as background, row (B) includes image patches correctly classified as containing Gram-positive chains/pairs, row (C) includes image patches correctly classified as containing Gram-positive clusters, and row (D) includes image patches correctly classified as containing Gram-negative rods. As described above in connection with FIG. 4, such images can be presented to a technologist to expedite smear review in a semi-automated process that uses the trained CNN to generate images for review that are likely to be diagnostically useful, allowing the technologist to focus on evaluating the images rather than attempting to find portions of the sample that are diagnostically useful.

The whole sample (whole slide) classification technique using the trained CNN was evaluated quantitatively, and compared to manual classification. Results are shown below in TABLE 1, which shows the manual classification of each slide, and corresponding prediction generated by a fully automated process to evaluate the sample on the slide using the trained CNN. As shown in TABLE 1, the fully automated process predicted the presence of bacteria in 84.7% of slides (i.e., 160 of the 189 slides) that were evaluated using the process. Of the slides in which the presence of bacteria was predicted, classification accuracy, sensitivity, and specificity are shown in TABLE 1. As shown in TABLE 1, observed classification accuracy was 92.5% across all categories, with a sensitivity of more than 97% for Gram-negative rods and Gram-positive clusters, and a relatively lower sensitivity for Gram-positive pairs or chains (which may be due to a relatively high number of misclassifications as Gram-positive clusters across a relatively low overall number of slides automatically classified as Gram-positive pairs or chains, with only 40 slides being classified as Gram-positive chains or pairs, and only 29 of the 189 slides including Gram-positive pairs or chains). Additionally, manual inspection of Gram-positive pairs or chains misclassified as Gram-positive clusters revealed that those slides were somewhat ambiguous, as substantial clumping of cells was present. Specificity for Gram-positive pairs or chains and Gram-negative rods was >96%. Specificity was slightly lower (93.2%) for Gram-positive clusters, which may be due to misclassification of Gram-positive pairs or chains as Gram-positive clusters. Despite qualitative differences in background staining, accuracy of slides from aerobic bottles (88.8%) or anaerobic bottles (92.9%) was not significantly different (using Fisher's exact test, P>0.05).

TABLE 1

| | Predicted Classification (n) | | | | | |
|---|---|---|---|---|---|---|
| | | Gram- | | | | |
| | | positive | Gram- | | | |
| Human | Gram- | pairs or | positive | | Sensitivity | Specificity |
| Classification | negative | chains | clusters | Background | % (CI) | % (CI)^{a} |
| Gram-negative | 51 | 1 | 0 | 17 | 98.1 | 96.3 |
| | | | | | (94.3-100) | (93.7-98.9) |
| Gram-positive | 3 | 27 | 6 | 4 | 75.0 | 98.4 |
| pairs or chains | | | | | (60.9-89.0) | (90.8-100) |
| Gram-positive | 1 | 1 | 70 | 8 | 97.2 | 93.2 |
| clusters | | | | | (93.4-100) | (89.7-96.6) |

(CI = 95% Confidence Interval)

The most common error in the whole sample classification test was misclassification of samples as background, representing 70.7% of all misclassifications (i.e., 29 of 41 misclassifications). A manual review of images from the slides that included misclassified samples found that 44.8% (i.e., 13 of 41 misclassifications) had insufficient image patches with bacteria to make a positive prediction based on the relatively high thresholds (i.e., a confidence of at least 0.99, and at least 6 image patches positively classified as belong to a particular class). An additional 48.3% (i.e., 14 of 41 misclassifications) included organisms that were out of focus or very low contrast, and of these, the majority (11 of 14, 78.6%) included Gram-negative organisms, which is an expected error based on the superficial similarity of Gram-negative cells bacteria and background material. The remaining 6.9% of misclassified samples (2 of 41 misclassifications) included highly elongated Gram-negative rods or minute Gram-negative coccobacilli, and neither morphology was included as an example in the training set. Gram stain category misclassifications other than conflation of Gram-positive cocci in chains and Gram-positive cocci in clusters (i.e., 5 of 41 misclassifications), were related to a combination of poor representation of the causal organism in image patches and excessive background artifacts.

For slides that were predicted to include cells, an overall classification accuracy of 92.5% was observed, and specificity of >93% for all classification labels with no human intervention. The most common classification error was misclassification of slides containing rare bacteria (e.g., containing bacteria, but not a type of bacteria the CNN was trained to recognize) as background, representing the majority (70.7%) of all classification errors. As described above in connection with FIG. 4, in a clinical setting the mechanisms described herein can flag samples classified as background for direct technologist review. Note that sensitivity/specificity in whole sample classification accuracy was lower compared to the sensitivity/specificity for per-image-patch classification. This may be due in part to inclusion of slides with very few bacteria in the set evaluated using the whole sample evaluation process, which may contribute to a greater propensity for false-positives.

The images used in training, verification, test, and evaluation of the trained CNN were taken from a total of 468 de-identified Gram-stained slides from positive blood cultures that were collected from the clinical microbiology laboratory at Beth Israel Deaconess Medical center between April and July, 2017 under an IRB-approved protocol. The slides were prepared during the course of normal clinical workup. No pre-selection of organism identity, organism abundance, or staining quality was performed prior to collection. Positive blood culture broth Gram stains included those prepared from both non-lytic, BD BACTEC Standard Aerobic (n=232) and lytic, BD BACTEC Lytic Anaerobic Medium (n=196) (BD, Sparks, Md.).

All slides were imaged without coverslips using a MetaFer Slide Scanning and Imaging platform with a 140-slide capacity automated slide loader equipped with a 40× magnification Plan-Neofluar objective (0.75 Numerical Aperture, available from Zeiss, of Oberkochen, Germany). For each slide, 54 images were collected from defined positions spanning the entirety of the slide. The first 279 slides collected were used in training, validation, testing, and evaluation of the trained CNN. The remaining 189 slides were manually re-classified as Gram-negative rods, Gram-positive chains/pairs or Gram-positive clusters using a Nikon Labophot 2 microscope (available from Nikon Inc., of Tokyo, Japan) equipped with a 100× oil objective. The results of the manual re-classification were recorded for use in evaluation of the whole-sample classification process.

A dataset of 146×146 pixel image patches was generated semi-autonomously using a Python script that facilitated patch selection, classification, and file archiving with a single input (e.g., mouse click), which allowed large numbers of labeled image patches to be saved in a relatively short period of time in a manner that was directly accessible to the CNN training program. Each image patch was labeled as belonging to one of four classifications: Gram-positive cocci in pairs or chains, Gram-positive cocci in clusters, Gram-negative rods, or background (no cells). Prior to training, the dataset was randomly divided into three subsets: 70% of image patches were assigned to a training set, 10% of image patches were assigned to an evaluation set for use during training, and 20% were assigned to a testing set to evaluate performance after completion of training. A transfer learning technique was used to re-train a portion of the Inception v3 CNN that was pre-trained on the ImageNet Large Scale Visual Recognition Competition (ILSVRC) 2012 image database. The Python language (version 3.5) and the TensorFlow library (version 1.0.1) were used to retrain the final fully connected layer of the CNN using a graphical user interface (GUI) controlling a modified script (“retrain.py”) from the TensorFlow GitHub repository. Training was performed using mini-batch gradient descent (batch size 200) with Nesterov momentum (momentum=0.9) and cross-entropy as the loss function. The initial learning rate was set at 0.001 and decayed exponentially at a rate of 0.99 per epoch. The output layer was a 4-way softmax classification layer which assigned probabilities to each of the four categories described above.

The trained CNN was evaluated on a per-image-patch basis using an evaluation set of 1,000 manually selected crops from each class (total crops=4,000), all of which were independent of the training set, validation set, and testing set. For each category, true positives were defined as image patches that were correctly classified as the category of interest; false positives were defined as image patches that were incorrectly classified as the category of interest; true negatives were defined as image patches that were correctly classified as a category other than the category of interest; and false negatives were defined as image patches that were incorrectly classified as a category other than the category of interest, where the category of interest was the label assigned to the image patch based on a manual classification. Sensitivity and specificity were modeled as ROC curves for each classification label by varying the softmax classification thresholds required for positivity. Sensitivity was defined as

$$\frac{True Positive}{True Positive + False Negative} .$$

Specificity was defined as

$$\frac{True Negative}{True Negative + False Positive} .$$

Area under the ROC curve was calculated for each label using the trapezium rule as implemented in the scipy library. The ROC curves shown in FIG. 7 were generated using the matplotlib library.

False positive rates for automatically selected image patches containing only background (no cells) were determined by analysis of 350 whole images (i.e., a single image captured at 40× magnification from a portion of a slide) from 40 different slides. Images contained no visible cells and were independent of the training set, validation set, testing set, and evaluation set. Each image was automatically segmented into 192 non-overlapping image patches of 146×146 pixels using a Python script (for a total of 67,200 image patches), and classified using the trained CNN using a stringent cutoff for positivity (cutoff=0.99). If no label achieved a probability greater than or equal to the cutoff, the associated crop was labeled as background. False positive rates were recorded for each classification label.

Using the fully automated imaging protocol described above for whole sample classification, whole sample classification accuracy was evaluated using images collected from 189 slides which were previously manually classified. For each slide, a Python script was employed to automatically divide each image of the 54 images collected from predefined locations on a slide into 192 image patches of 146×146 pixels. Each image patch was evaluated using the trained CNN which assigned probabilities for each category (Gram-negative rods, Gram-positive chains/pairs, Gram-positive clusters, and background), and a stringent cutoff was used for assigning a classification (cutoff=0.99), such that if no label met the classification cutoff, the image was classified as background.

After classification of all image patches collected from a particular slide, the sample was labeled as the class for which the greatest number of image patches were predicted to be members of that class. However, the sample was only labeled as a particular class if the number of image patches predicted to be members of that class exceeded the number of expected false positives (e.g., as described above). If none of the three label categories representing organisms were selected based on the criteria, the sample was classified as background. Results were recorded and used to construct a confusion matrix tabulation. Whole-sample sensitivity and specificity were defined and calculated. Classification accuracy for slides from aerobic or anaerobic bottles were compared using Fisher's exact test with significance defined as P<0.05 (JMP Pro version 13.0).

In general, CNNs show improved performance as more examples are included in the training set. However, unlike some other machine learning models, using more examples (i.e., more training data) does not increase the size of a CNN, and does not increase the complexity of implementation, as the total number of layers and parameters is typically fixed based on the design of the CNN. Training an entire CNN does require substantial computational infrastructure, but using transfer learning to modify an existing trained CNN can reduce the total amount of training time. For example, using transfer learning, a version of the Inception v3 CNN was trained and implemented using a personal computer having an Intel Core i7 CPU, 32 GB RAM, and no GPU. However, using this computing device for whole sample analysis using the trained CNN was relatively slow due to the relatively large number of image patches that were classified for each sample (i.e., 10,368 image patches analyzed per slide). Using more suitable and/or powerful computing equipment can result in reductions in the amount of time required to perform the fully automated whole sample evaluation described above. For example, using a computing device including an Nvidia GTX 1070 GPU whole sample classification time was reduced by a factor of 6 in comparison to performing the same process using the personal computer having the Intel Core i7 CPU and no GPU (e.g., a classification time of ˜9 minutes, rather than a classification time on the order of one hour). This may be further reduced by using a more powerful GPU, or techniques for executing portions of the process in parallel.

Note that Gram stain smear preparation may have a significant impact on automated slide imaging. In the tests described herein, slides were prepared by technologists during the course of normal laboratory operation, and exhibited a relatively high degree of variability in smear area, thickness, location, and staining intensity. Standardization of such variables may improve the ability of an automated microscope to consistently sample microscopic fields with evaluable organisms. Further, use of an automated Gram stain device for staining may also increase reproducibility of staining characteristics and may increase accuracy.

As described above in connection with FIG. 4, the mechanisms described herein can be used as part of a semi-automated process to augment technologist classification. In general, manual interpretation of blood culture Gram stains by trained technologists are very accurate. The mechanisms described herein can enhance productivity of such technologists by selectively presenting images from a sample that are predicted to contain bacteria to local and/or remotely located technologists. This may spare the technologist from the need to manually locate fields of interest among a preponderance of background. This may also reduce overall technologist read time (e.g., from minutes per slide to on the order of seconds). Additionally or alternatively, the mechanisms described herein can be used as part of a fully automated classification platform with no human intervention required to classify a sample.

Additionally, in some embodiments, the mechanisms described herein can be configured to train CNNs for other smear-based biological diagnostics, such as those used in clinical diagnostic laboratories as well as specialized parasitology, mycobacteriology, and mycology laboratories. In some embodiments, the mechanisms described herein can be configured to classify organism morphology (e.g., cocci, rods, coccobacilli, coryneform, Bacillus/Clostridium-like, cocci in pairs, clusters, chains and/or tetrads) of organisms present in a positive blood culture Gram stain slide. In some embodiments, such morphology classification can aid in rapid definition of the type of organism in positive blood cultures, which can be used to guide therapeutic decisions, such as determining whether a patient is on appropriate therapy.

In some embodiments, the mechanisms described herein can be configured to evaluate a liquid biological sample (e.g., sputum, saliva, bronchoalveolar lavage fluid, lymph, urine, pleural fluid, ascites fluid, synovial fluid, fluids obtained by irrigating or washing various body cavities, etc.) prepared using Gram stain. In some cases, the liquid biological sample is obtained by grinding or otherwise breaking down a solid biological tissue specimen, resuspending the ground material in a buffer (e.g., saline), and the suspended ground material is applied to a slide for Gram staining.

In certain embodiments, the mechanisms described herein can be configured to classify organism morphology (e.g., as described above in connection with positive blood culture Gram stain slides) of organisms present in a liquid biological sample. In some cases, the mechanisms are configured to classify organism morphology or organisms present in a sputum or bronchoalveolar lavage fluid sample prepared for analysis using Gram stain. In some embodiments, using a trained CNN to evaluate one or more properties of a liquid biological sample comprising sputum or bronchoalveolar lavage fluid prepared using a Gram stain can, for example, provide rapid therapeutic guidance for conditions such as community acquired pneumonia (CAP), hospital acquired pneumonia (HAP), and ventilator associated pneumonia (VAP).

As another example, the mechanisms described herein can be configured to determine the number and/or morphological features of squamous epithelial cells in the sample to assess quality of sputa. In such cases, the smear of liquid biological sample is Gram stained for training and interpretation. As yet another example, the mechanisms described herein can be configured to determine in a sample the number and/or morphological features of neutrophils (also known as polymorphonuclear leucocytes), which are white blood cells specialized in the fight against micro-organisms by phagocytosis, as an indication of the presence and/or degree of inflammation.

In some embodiments, the mechanisms described herein can be configured to evaluate a fixed tissue slice prepared using tissue Gram stain. For example, the mechanisms described herein can be configured to detect the presence of organisms in a fixed tissue slice prepared using tissue Gram stain. As another example, the mechanisms described herein can be configured to classify organism morphology (e.g., as described above in connection with positive blood culture Gram stain slides) of organisms present in a fixed tissue slice prepare using tissue Gram stain. As yet another example, the mechanisms described herein can be configured to determine the number and/or morphology of neutrophils in the fixed tissue slice prepared using tissue Gram stain to assess degree of inflammation. Similarly, the mechanisms described herein can be configured to determine the number and/or morphology of other inflammatory cells including, without limitation, macrophages, lymphocytes, and plasma cells in the fixed tissue slice prepared using Tissue Gram stain in order to produce additional information about potential infectious agents and chronicity of infection. In some embodiments, using a trained CNN to evaluate one or more properties of a fixed tissue slice prepared using tissue Gram stain can, for example, facilitate detection of organisms directly in tissue, or identification of infection without culturing the organism.

In some embodiments, the mechanisms described herein can be configured to evaluate a sputum or bronchoalveolar lavage fluid sample prepared using acid-fast stain (e.g., using auromine-rhodamine). As used herein, the term “acid-fast” refers to carbolfuchsin or auromine-rhodamine staining reactions for certain types of bacilli, such as mycobacteria which resist destaining by acids or alcohol. For example, the mechanisms described herein can be configured to detect the presence of acid-fast organisms (e.g., acid-fast bacilli) in the sample with fluorescent stain. In some embodiments, using a trained CNN to evaluate one or more properties of a sputum or bronchoalveolar lavage fluid sample prepared using acid-fast stain can, for example, provide a rapid screen for sputa that contains mycobacterium, or provides rapid information on potential infectivity of patients suspected of being infected with Mycobacterium tuberculosis.

In some embodiments, the mechanisms described herein can be configured to evaluate a Löwenstein-Jensen medium or Middlebrook broth with presumptive mycobacteria prepared using acid-fast stain (e.g., using a Ziehl-Neelsen or Kinyoun method). For example, the mechanisms described herein can be configured to detect the presence of acid-fast organisms (e.g., acid-fast bacilli) in culture. As another example, the mechanisms described herein can be configured to detect the presence of cording phenotype characteristic of Mycobacterium tuberculosis in the sample. In some embodiments, using a trained CNN to evaluate one or more properties of a Löwenstein-Jensen medium or Middlebrook broth with presumptive mycobacteria prepared using acid-fast stain can, for example, identify the presence of mycobacteria in culture.

In some embodiments, the mechanisms described herein can be configured to evaluate a sputum, bronchoalveolar lavage fluid, or positive blood culture broth sample prepared using fluorescent probes. For example, the mechanisms described herein can be configured to identify the species of organisms in the sample based on specific fluorescent probes for ribosomal RNA or DNA. In some embodiments, using a trained CNN to evaluate one or more properties of a sputum or bronchoalveolar lavage fluid sample prepared using fluorescent probes can, for example, facilitate identification of the species of an organism directly from specimen, and can be used in combination with Gram stain analysis to provide highly specific interpretation to guide therapy.

In some embodiments, the mechanisms described herein can be configured to evaluate a stool sample prepared using fluorescent probes. For example, the mechanisms described herein can be configured to detect common stool parasites in the stool sample, such as Cryptosporidium or Giardia. In some embodiments, using a trained CNN to evaluate one or more properties of a stool sample prepared using fluorescent probes can, for example, facilitate identification of common stool parasites without requiring a complete ova and parasite exam.

In some embodiments, the mechanisms described herein can be configured to evaluate a stool sample prepared using iodine or saline wet preparation techniques to perform an ova and parasite exam. For example, the mechanisms described herein can be configured to detect and classify eggs and protozoa present in the stool sample prepared using iodine or saline wet preparation techniques. In some embodiments, using a trained CNN to evaluate one or more properties of a stool sample prepared using iodine or saline wet preparation techniques can, for example, facilitate identification of stool parasites, which is typically an extremely labor intensive and operator dependent test.

In some embodiments, the mechanisms described herein can be configured to evaluate a stool sample prepared using trichrome stain, iron hematoxylin or modified iron hematoxylin to perform an ova and parasite exam. For example, the mechanisms described herein can be configured to detect and classify eggs and protozoa present in the stool sample prepared using iodine or saline wet preparation techniques. In some embodiments, using a trained CNN to evaluate one or more properties of a stool sample prepared using one or more of a trichrome stain, iron hematoxylin, or modified iron hematoxylin can, for example, facilitate identification of stool parasites, which is typically an extremely labor intensive and operator dependent test.

In some embodiments, the mechanisms described herein can be configured to evaluate a sample of cerebrospinal fluid (CSF) prepared using Gram stain of a cytospin smear (e.g., a slide prepared by a cytospin technique) or direct Gram stain. For example, the mechanisms described herein can be configured to detect and categorize pathogens in the sample of cerebral spinal fluid based on size, shape, and/or morphology of organisms, such as bacterial causes of meningitis or fungal causes of meningitis. In some embodiments, using a trained CNN to evaluate one or more properties of a sample of cerebrospinal fluid prepared using Gram stain of a cytospin smear or direct Gram stain can, for example, facilitate identification of bacterial and fungal causes of meningitis which can allow lifesaving application of specific therapy. CSF samples for use with the mechanisms described herein may be obtained from a subject by any suitable method, including but not limited to withdrawing CSF from a subject using a transcutaneous catheter and external collection bag or other receptacle.

In some embodiments, the mechanisms described herein can be configured to evaluate a blood parasite smear prepared using Giemsa stain or Wright-Giemsa to perform a test for blood parasites. For example, the mechanisms described herein can be configured to detect and speciate Babesia and Plasmodium species in a sample of blood. The terms “Wright-Giemsa stain” and “Giemsa stain” generally refer to solutions comprising thiazin compounds (e.g., chemical substances comprising a ring of four carbon atoms, one nitrogen atom, and one sulfur atom) and eosin compounds (e.g., chemical substances comprising brominated fluorescein) in various concentrations. Such solutions may be commercially available in concentrated or diluted form, or may be created by dissolving a specified amount of thiazin and/or eosin compounds in a suitable solvent to create such a solution. As another example, the mechanisms described herein can be configured to quantify Babesia and Plasmodium parasites to calculate a percent parasitemia in a sample of blood. In some embodiments, using a trained CNN to evaluate one or more properties of a blood parasite smear prepared using Giemsa stain or Wright-Giemsa can, for example, facilitate rapid identification and differentiation among Babesia and Plasmodium species which can aid in making treatment decisions to provide potentially life-saving therapy. Additionally or alternatively, in some embodiments, using a trained CNN to evaluate one or more properties of a blood parasite smear prepared using Giemsa stain or Wright-Giemsa can facilitate calculation of percent parasitemia for sequential smears, which can be used to document response to therapy, and may indicate need for adjustment to therapy or the need for extra interventions, such as red blood cell exchange.

In some embodiments, the mechanisms described herein can be configured to evaluate a hematology smear stained using mixtures of thiazin and eosin dyes. Such stains include, without limitation, Wright-Giemsa stains and May-Grunwald stains. For example, the mechanisms described herein can be configured to detect, and/or classify a subtype of, leukemia in a hematology smear. The term “May-Grunwald stain” generally refers to solutions comprising thiazin compounds (e.g., chemical substances comprising a ring of four carbon atoms, one nitrogen atom, and one sulfur atom) and eosin compounds (e.g., chemical substances comprising brominated fluorescein) in various concentrations. May-Grunwald stains may be commercially available in concentrated or diluted form, or may be created by dissolving a specified amount of thiazin and/or eosin compounds in a suitable solvent to create such a solution. In some embodiments, using a trained CNN to evaluate one or more properties of a hematology smear prepared using Wright-Giemsa can, for example, detect the presence of leukemia, which can be a rapidly progressing, life threatening illness. Additionally or alternatively, in some embodiments, using a trained CNN to evaluate one or more properties of a hematology smear prepared using Wright-Giemsa can facilitate identification of subtypes of leukemia, which may require specific therapies. Rapid morphological diagnosis using n semi-autonomous or fully autonomous process can expedite diagnosis, treatment, and triage for patients with leukemia.

Blood samples for use according to any of the mechanisms described herein may be obtained from a subject by any suitable method, including but not limited to withdrawing blood from a subject using a needle and syringe. In some embodiments, blood samples may be collected from a subject using a finger-stick technique, wherein the subject's skin is punctured and a sufficient amount of blood is “milked” or expressed from the puncture. Blood samples suitable for use in the subject methods include both venous and arterial blood samples. Any blood sample collection procedure may be readily adapted for use with the subject methods.

In some embodiments, the mechanisms described herein can be configured to evaluate a sample including fungal hyphae, reproductive structures, and/or spores prepared using lactophenol cotton blue. Samples may be taken from fungal colonies on an agar plate (such as Sabouraud dextrose agar), transferred to slides, and stained using lactophenol cotton blue or imaged without preparatory staining using phase-contrast optics. Samples may also represent histologic sections stained with Gomori's methenamine silver stain (GMS), Fontana-Masson, Mucicarmine, H&E, and/or PAS stains. For example, the mechanisms described herein can be configured to identify fungal genus and/or species. In some embodiments, using a trained CNN to evaluate one or more properties of a sample including fungal hyphae, reproductive structures, and/or spores prepared using lactophenol cotton blue can, for example, identify fungal morphology, which can facilitate identification, but which typically requires a very highly trained technologist for interpretation. In some embodiments, such identification can be performed based on automatically collected images or in “real time” with technologist input.

It should be noted that, as used herein, the term mechanism can encompass hardware, software, firmware, or any suitable combination thereof.

It should be understood that, with the exception of CNN training which must follow receipt of images as training data, the above described steps of the processes of FIG. 4 can be executed or performed in any order or sequence not limited to the order and sequence shown and described in the figures. Also, some of the above steps of the processes of FIG. 4 can be executed or performed substantially simultaneously where appropriate or in parallel to reduce latency and processing times.

Although the invention has been described and illustrated in the foregoing illustrative embodiments, it is understood that the present disclosure has been made only by way of example, and that numerous changes in the details of implementation of the invention can be made without departing from the spirit and scope of the invention, which is limited only by the claims that follow. Features of the disclosed embodiments can be combined and rearranged in various ways.

Each of the following references is hereby incorporated herein by reference in its entirety:
Laupland K B. 2013. Incidence of bloodstream infection: a review of population-based studies. Clin Microbiol Infect 19:492-500.
Wisplinghoff H, Bischoff T, Tallent S M, Seifert H, Wenzel R P, Edmond M B. 2004. Nosocomial bloodstream infections in US hospitals: analysis of 24,179 cases from a prospective nationwide surveillance study. Clin Infect Dis 39:309-17.
Schwaber M J, Carmeli Y. 2007. Mortality and delay in effective therapy associated with extended-spectrum beta-lactamase production in Enterobacteriaceae bacteraemia: a systematic review and meta-analysis. J Antimicrob Chemother 60:913-20.
Kang C I, Kim S H, Kim H B, Park S W, Choe Y J, Oh M D, Kim E C, Choe K W. 2003. Pseudomonas aeruginosa bacteremia: risk factors for mortality and influence of delayed receipt of effective antimicrobial therapy on clinical outcome. Clin Infect Dis 37:745-51.
Wain J, Diep T S, Ho V A, Walsh A M, Nguyen T T, Parry C M, White N J. 1998. Quantitation of bacteria in blood of typhoid fever patients and relationship between counts and clinical features, transmissibility, and antibiotic resistance. J Clin Microbiol 36:1683-7.
Barenfanger J, Graham D R, Kolluri L, Sangwan G, Lawhorn J, Drake C A, Verhulst S J, Peterson R, Moja L B, Ertmoed M M, Moja A B, Shevlin D W, Vautrain R, Callahan C D. 2008. Decreased mortality associated with prompt Gram staining of blood cultures. Am J Clin Pathol 130:870-6.
Bourbeau P P, Ledeboer N A. 2013. Automation in clinical microbiology. J Clin Microbiol 51:1658-65.
Garcia E, Ali A M, Soles R M, Lewis D G. 2015. The American Society for Clinical Pathology's 2014 vacancy survey of medical laboratories in the United States. Am J Clin Pathol 144:432-43.
Meyer J, Pare G. 2015. Telepathology Impacts and Implementation Challenges: A Scoping Review. Arch Pathol Lab Med 139:1550-7.
LeCun Y, Bengio Y, Hinton G. 2015. Deep learning. Nature 521:436-44.
Smith K P, Richmond D L, Brennan-Krohn T, Elliott H L, Kirby J E. 2017. Development of MAST: A Microscopy-Based Antimicrobial Susceptibility Testing Platform. SLAS Technol doi:10.1177/2472630317727721:2472630317727721.
Szegedy C, Wei L, Yangqing J, Sermanet P, Reed S, Anguelov D, Erhan D, Vanhoucke V, Rabinovich A. Going deeper with convolutions, p 1-9. In (ed).
Shin H-C, Roth H R, Gao M, Lu L, Xu Z, Nogues I, Yao J, Mollura D, Summers R M. 2016. Deep Convolutional Neural Networks for Computer-Aided Detection: CNN Architectures, Dataset Characteristics and Transfer Learning. IEEE Transactions on Medical Imaging 35:1285-1298.
Russakovsky O, Deng J, Su H, Krause J, Satheesh S, Ma S, Huang Z, Karpathy A, Khosla A, Bernstein M, Berg A C, Fei-Fei L. 2015. ImageNet Large Scale Visual Recognition Challenge. International Journal of Computer Vision 115:211-252.
Bridle J S. 1989. Probabilistic Interpretation of Feedforward Classification Network Outputs, with Relationships to Statistical Pattern Recognition. Neurocomputing F68:227-236.
Esteva A, Kuprel B, Novoa R A, Ko J, Swetter S M, Blau H M, Thrun S. 2017. Dermatologist-level classification of skin cancer with deep neural networks. Nature 542:115-118.
Madani A, Arnaout R, Mofrad M, Arnaout R. 2017. Fast and accurate classification of echocardiograms using deep learning. https(colon)//arxiv(dot)org/ftp/arxiv/papers/1706/1706.08658.pdf. Accessed Sep. 22, 2017.
Litjens G, Sanchez C I, Timofeeva N, Hermsen M, Nagtegaal I, Kovacs I, Hulsbergen-van de Kaa C, Bult P, van Ginneken B, van der Laak J. 2016. Deep learning as a tool for increased accuracy and efficiency of histopathological diagnosis. Sci Rep 6:26286.
Wang D, Khosla A, Gargeya R, Irshad H, Beck A H. 2016. Deep Learning for Identifying Metastatic Breast Cancer. https(colon)//arxiv(dot)org/abs/1606.05718. Accessed Sep. 12, 2017.
Samuel L P, Balada-Llasat J M, Harrington A, Cavagnolo R. 2016. Multicenter Assessment of Gram Stain Error Rates. J Clin Microbiol 54:1442-7.
Sogaard M, Norgaard M, Schonheyder H C. 2007. First notification of positive blood cultures and the high accuracy of the gram stain report. J Clin Microbiol 45:1113-7.
Rand K H, Tillan M. 2006. Errors in interpretation of Gram stains from positive blood cultures. Am J Clin Pathol 126:686-90.
Anonymous. 2017. TensorFlow: An open-source software library for Machine Intelligence https(colon)//www(dot)tensorflow(dot)org/. Accessed Sep. 12, 2017.
Anonymous. 2017. TensorFlow GitHub Repository. https(colon)//github(dot)com/tensorflow/tensorflow. Accessed Sep. 12, 2017.
Nesterov Y. 1983. A method of solving a convex programming problem with convergence rate O(1/sqr(k)). Soviet Mathematics Doklady 27:372-376.
Jones E, Oliphant E, Peterson P. 2001. SciPy: Open Source Scientific Tools for Python. http(colon)//www(dot)scipy(dot)org/. Accessed Sep. 12, 2017.
Hunter J D. 2007. Matplotlib: A 2D graphics environment. Computing in Science and Engineering 9:90-95.
Stehman S V. 1997. Selecting and interpreting measures of thematic classification accuracy. Remote Sensing of Environment 62:77-89.

1. A method for automatically classifying a biological sample, comprising:
receiving an image of the biological sample;
extracting a plurality of image patches from the image;
providing each of the plurality of image patches to a trained convolution neural network that was trained using transfer learning and a plurality of images of pre-classified and labeled biological samples;
receiving, for each of the plurality of image patches, a classification from the trained convolution neural network indicating a prediction that the image patch includes an example of a particular class of a plurality of classes;
counting, for each of the plurality of classes, the number of image patches predicted by the trained classifier to include examples of the class; and
classifying, based on the count for each of the plurality of classes, the biological sample.

2. The method of claim 1, wherein the biological sample is a Gram-stained blood culture slide.

3. The method of claim 2, wherein the plurality of classes includes at least a first class corresponding to Gram-negative rods, a second class corresponding to Gram-positive cocci in clusters, and a third class corresponding to Gram-positive cocci in pairs and chains.

4. The method of claim 2, wherein the plurality of classes includes one or more classes selected from the group consisting of Gram-negative cocci in pairs, Gram-negative coccobacilli, Gram-positive coccobacilli, Gram positive cocci in pairs, Gram-positive diptheroidal morphologies, Gram-positive bacilli/clostridial morphologies, and Gram-positive rods in chains.

5. The method of claim 1, wherein the trained convolution neural network is a version of an Inception v3 convolution neural network.

6. The method of claim 5, wherein the Inception v3 convolution neural network was trained to classify a multitude of different object classes from input image data using labeled images including examples of the different object classes, wherein each of the plurality of classes is different than all of the different object classes.

7. The method of claim 6, wherein a final fully connected layer of the Inception v3 convolution neural network was retrained using the plurality of images of pre-classified and labeled biological samples.

8. The method of claim 1, wherein the biological sample comprises at least one of the following liquids: blood, plasma, serum, sputum, saliva, bronchoalveolar lavage fluid, lymph, urine, pleural fluid, ascites fluid, synovial fluid, and fluid obtained by irrigating or washing a body cavity.

9. (canceled)

10. The method of claim 8, wherein the plurality of classes includes at least a first class corresponding to the presence of a first organism, and a second class corresponding to absence of the first organism.

11. The method of claim 8, wherein the biological sample comprises sputum, wherein the plurality of classes includes at least a first class corresponding to the presence of a squamous epithelial cell, and wherein the method further comprises determining the prevalence of squamous epithelial cells in the sputum.

12. The method of claim 1, wherein the biological sample comprises a fluid prepared using an acid-fast stain.

13. The method of claim 12, wherein the fluid comprises sputum or bronchoalveolar lavage fluid.

14. The method of claim 13, wherein the plurality of classes includes at least a first class corresponding to the presence of mycobacteria, and a second class corresponding to the absence of mycobacteria.

15. The method of claim 1, wherein the biological sample comprises Löwenstein-Jensen medium or Middlebrook broth with presumptive mycobacteria prepared using acid-fast stain.

16. The method of claim 15, wherein the plurality of classes includes at least a first class corresponding to the presence of one or more acid-fast organisms.

17. The method of claim 1, wherein the biological sample comprises a fluid sample prepared using fluorescent probes.

18. The method of claim 17, wherein the fluid comprises sputum or bronchoalveolar lavage fluid.

19. The method of claim 18, wherein the plurality of classes includes at least a first class corresponding to the presence of a first type of organism, and a second class corresponding to the presence of a second organism.

20. The method of claim 1, wherein the biological sample comprises a stool sample prepared using fluorescent probes.

21. The method of claim 20, wherein the plurality of classes includes at least a first class corresponding to the presence of Cryptosporidium or Giardia, and a second class corresponding to the absence of Cryptosporidium or Giardia.

22. The method of claim 1, wherein classifying the biological sample comprises classifying the biological sample based on the class having the highest count or classifying the biological sample based on one or more classes having a count over a threshold.

23. (canceled)

24. The method of claim 22, wherein each of the one or more classes is associated with a threshold, and the threshold for a first class is different than a threshold for a second class.
